(19) United States
(12) Patent Application Publication
(10) Pub. No.: US 2024/0206818 A1
(43) Pub. Date: Jun. 27, 2024

(54) MEASUREMENT DEVICE

(71) Applicant: OSAKA UNIVERSITY, Osaka (JP)

(72) Inventors: Teruo KANKI, Osaka (JP); Yasushi HOTTA, Hyogo (JP)

(21) Appl. No.: 18/288,867

(22) PCT Filed: Apr. 22, 2022
(86) PCT No.: PCT/JP2022/018600

§ 371 Date: Oct. 30, 2023

(30) Foreign Application Priority Data
May 12, 2021 (JP) 2021-081120

(51) Int. Cl. A61B 5/00; A61B 5/308; A61B 5/31; A61B 5/313

(52) U.S. Cl. CPC A61B 5/7203; A61B 5/308; A61B 5/31; A61B 5/313

(57) ABSTRACT

A measurement device includes: a first electrode; a second electrode; a ground electrode disposed between the first electrode and the second electrode; a differential circuit configured to generate a differential voltage between a first voltage input from the first electrode and a second voltage input from the second electrode; a ground terminal connected to each of the ground electrode, the differential circuit, and an output circuit; the output circuit including an input terminal to which the differential voltage is input, a reference terminal to which a reference voltage is input, and a common terminal, the output circuit being configured to generate a measurement signal based on the differential voltage and the reference voltage, and to output the generated measurement signal; and an impedance element including a first end connected to each of the ground terminal and the common terminal, and a second end connected to the reference terminal.

### TECHNICAL FIELD

The present invention relates to a measurement device that measures a biosignal.

### BACKGROUND ART

In recent years, research has been conducted on a measurement device that measures a biosignal of a person during exercise, such as jogging, in real time. Non-Patent Literature 1 discloses a measurement circuit including a negative electrode attached to the left wrist of a person, a positive electrode attached to the right wrist of the person, an amplifier that outputs a potential difference between the potential of the positive electrode and the potential of the negative electrode, and a ground electrode attached to the ankle to stabilize the potential difference.

However, with the measurement circuit described in Non-Patent Literature 1, it is difficult to measure an electrocardiographic signal of a person during the person's daily activities because the ground electrode is attached to the ankle. In addition, according to Non-Patent Literature 1, it is difficult to detect the electrocardiographic signal accurately because removal of a noise signal including an electromyographic signal, a body surface potential signal, and an internal potential signal from the measured electrocardiographic signal is not disclosed at all. Here, a method of removing the noise signal mixed in the measured electrocardiographic signal by signal processing is also conceivable. However, this requires a separate signal processing circuit to be provided in the electrocardiograph, leading to an increase in the size and cost of the electrocardiograph.

### CITATION LIST

### Non Patent Literature

Non-Patent Literature 1: Nikkei xTECH: Measure your heart rate just by wearing a device, what is the ability of the device? [online], Feb. 5, 2016, [searched Apr. 2, 2021], Internet, <URL:https://xtech.nikkei.com/dm/atcl/column/15/110200016/122100017/?P=4>

### SUMMARY OF INVENTION

The present invention has been made to solve such a problem, and an object of the present invention is to provide a measurement device that can remove the noise signal accurately even with a simple configuration.

A measurement device according to one aspect of the present invention is a measurement device for measuring a biosignal, including: a first electrode disposed in a measurement area of a body according to the biosignal to be measured; a second electrode disposed in the measurement area; a ground electrode disposed between the first electrode and the second electrode in the measurement area; a differential circuit configured to generate a differential voltage between a first voltage input from the first electrode and a second voltage input from the second electrode; an output circuit including an input terminal to which the differential voltage is input, a reference terminal to which a reference voltage is input, and a common terminal, the output circuit being configured to generate a measurement signal based on the differential voltage and the reference voltage, and to output the generated measurement signal; a ground terminal connected to each of the ground electrode, the differential circuit, and the output circuit; and an impedance element including a first end connected to each of the ground terminal and the common terminal, and a second end connected to the reference terminal.

According to the present invention, the noise signal can be removed accurately even with a simple configuration.

### BRIEF DESCRIPTION OF DRAWINGS

FIG. 1 is a circuit diagram showing one example of a configuration of a measurement device according to an embodiment of the present disclosure.

FIG. 2 is a diagram showing an example of attaching a first electrode, a second electrode, and a ground electrode to a human.

FIG. 3 is a diagram showing a first example of a measurement area.

FIG. 4 is a diagram showing a second example of the measurement area.

FIG. 5 is a diagram showing a mounting example of the first terminal, the second terminal, and the ground terminal.

FIG. 6 is a graph showing an experimental result when measuring an electrocardiographic signal of a subject in a resting state by using a measurement device of a comparative example.

FIG. 7 is a graph showing an experimental result when measuring the electrocardiographic signal of the subject in a resting state by using the measurement device of the present embodiment.

FIG. 8 is a graph showing an experimental result when measuring the electrocardiographic signal of a subject in a jogging state by using the measurement device of the comparative example.

FIG. 9 is a graph showing an experimental result when measuring the electrocardiographic signal of the subject in a jogging state by using the measurement device of the present embodiment.

FIG. 10 is a graph showing an experimental result when measuring the electrocardiographic signal of a subject in a running state by using the measurement device with impedance of an impedance element of 0Ω.

FIG. 11 is a graph showing an experimental result when measuring the electrocardiographic signal of the subject in a running state by using the measurement device with impedance of the impedance element of 1.0 MΩ.

FIG. 12 is a graph showing an experimental result when measuring the electrocardiographic signal of the subject in a running state by using the measurement device with impedance of the impedance element of 3.9 MΩ.

FIG. 13 is a graph showing an experimental result when measuring the electrocardiographic signal of the subject in a running state by using the measurement device with impedance of the impedance element of ∞Ω.

FIG. 14 is a graph showing an experimental result when measuring the electrocardiographic signal of a subject in an arm-swinging state by using the measurement device with impedance of the impedance element of 0Ω.

FIG. 15 is a graph showing an experimental result when measuring the electrocardiographic signal of the subject in an arm-swinging state by using the measurement device with impedance of the impedance element of 1.0Ω.

FIG. 16 is a graph showing an experimental result when measuring the electrocardiographic signal of the subject in an arm-swinging state by using the measurement device with impedance of the impedance element of 3.9 MΩ.

FIG. 17 is a graph showing an experimental result when measuring the electrocardiographic signal of the subject in an arm-swinging state by using the measurement device with impedance of the impedance element of ∞Ω.

FIG. 18 is a diagram showing an example of attaching the first electrode, the second electrode, and the ground electrode when the measurement area is the back.

FIG. 19 is a diagram showing the first electrode, the second electrode, and the ground electrode attached to a bathtub.

FIG. 20 is a diagram showing a human while taking a bath in the bathtub.

FIG. 21 is a diagram showing the electrocardiographic signal of the human while taking a bath.

### DESCRIPTION OF EMBODIMENTS

An embodiment of the present invention will be described below with reference to the accompanying drawings. Note that the following embodiment is one example of embodying the present invention, and does not limit the technical scope of the present invention in nature.

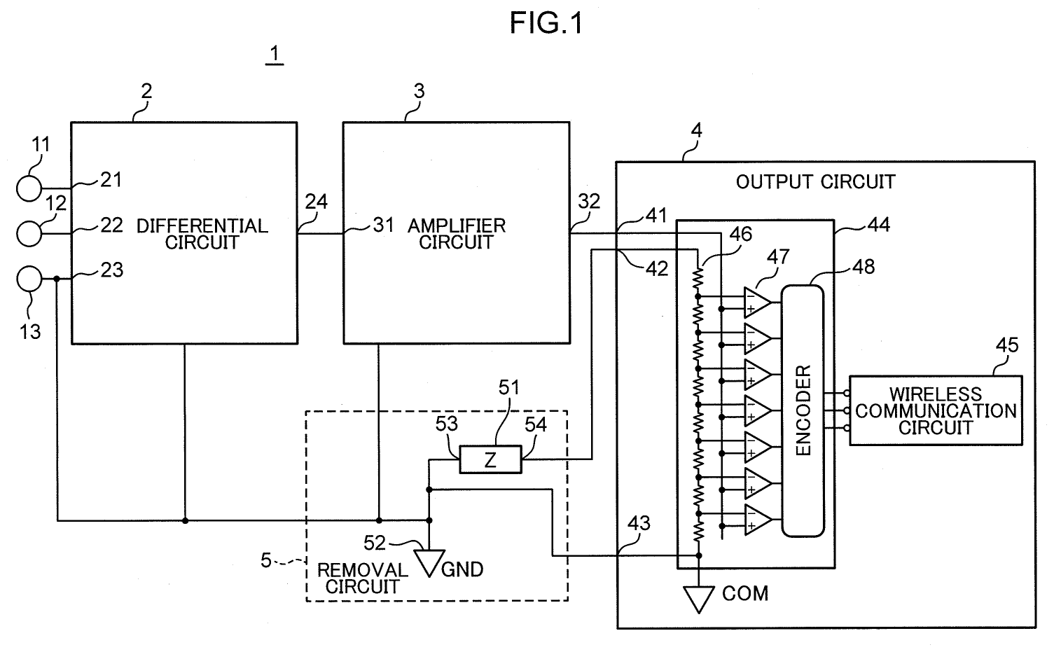

FIG. 1 is a circuit diagram showing one example of the configuration of a measurement device 1 according to the embodiment of the present disclosure. The measurement device 1 is a device that measures a biosignal. The biosignal is, for example, a biosignal of a human body. Hereinafter, the biosignal to be measured is referred to as a ‘target biosignal’. The target biosignal is described as an electrocardiographic signal. However, this is one example, and the target biosignal may be an electromyographic signal or an electroencephalographic signal.

The measurement device 1 includes a first electrode 11, a second electrode 12, a ground electrode 13, a differential circuit 2, an amplifier circuit 3, an output circuit 4, and a removal circuit 5. The first electrode 11 is disposed in a measurement area of the body according to the target biosignal. Here, since the target biosignal is the electrocardiographic signal, the measurement area is an area near the heart, for example, an area of the upper body. The second electrode 12 is disposed in the measurement area. The ground electrode 13 is disposed between the first electrode 11 and the second electrode 12 in the measurement area.

The differential circuit 2 generates a differential voltage between a first voltage input from the first electrode 11 and a second voltage input from the second electrode 12. The differential circuit 2 includes an input terminal 21, an input terminal 22, a ground terminal 23, and an output terminal 24. The input terminal 21 is connected to the first electrode 11, and the first voltage is input. The input terminal 22 is connected to the second electrode 12, and the second voltage is input. The ground terminal 23 is connected to the ground electrode 13 and a ground terminal 52. The output terminal 24 is connected to an input terminal 31 of the amplifier circuit 3. The differential circuit 2 may have, for example, a function of a high-pass filter that allows signals with frequencies of a predetermined cut-off frequency or higher to pass through and/or a function of amplifying an input signal. For example, as the differential circuit 2, a differential amplifier circuit may be employed, or an instrumentation amplifier circuit may be employed.

The amplifier circuit 3 amplifies the differential voltage and inputs the amplified differential voltage into the output circuit 4. The amplifier circuit 3 includes the input terminal 31 and an output terminal 32. The output terminal 32 is connected to an input terminal 41 of the output circuit 4. The amplifier circuit 3 includes, for example, a known amplifier circuit including an operational amplifier. The amplifier circuit 3 may have a function of a low-pass filter that allows voltages with frequencies of a predetermined cutoff frequency or lower to pass through and/or a function of inverting and outputting the voltage input into the input terminal 31. The amplifier circuit 3 amplifies the differential signal, for example, with a predetermined gain suitable for an analog-to-digital converter 44 to convert the differential signal into an analog-to-digital signal.

The ground terminal 52 is connected to each of the ground electrode 13, the differential circuit 2, and the amplifier circuit 3, and sets a reference potential for the ground electrode 13, the differential circuit 2, and the amplifier circuit 3. The reference potential is, for example, 0 V.

The output circuit 4 includes the input terminal 41, a reference terminal 42, a common terminal 43, the analog-to-digital converter 44, and a wireless communication circuit 45. The input terminal 41 is connected to the output terminal 32, and the differential voltage is input. A reference voltage is input into the reference terminal 42. The common terminal 43 is a ground terminal of the output circuit 4 and sets the reference potential for the output circuit 4. The output circuit 4 generates a measurement signal based on the differential voltage and the reference voltage, and outputs the generated measurement signal.

The analog-to-digital converter 44 is, for example, a flash-type analog-to-digital converter. However, this is one example, and the analog-to-digital converter 44 may include a pipeline type, a successive approximation type, or a delta-sigma type analog-to-digital converter. The analog-to-digital converter 44 includes a ladder resistance unit 46, a comparator unit 47, and an encoder 48. In the ladder resistance unit 46, a first end is connected to the reference terminal 42, and a second end is connected to the common terminal 43. The ladder resistance unit 46 divides the reference voltage input into the reference terminal 42 into 2n−1 voltages (n is an integer equal to or greater than 2). In the comparator unit 47, a first end is connected to the input terminal 41. The comparator unit 47 simultaneously compares each of the divided 2n−1 reference voltages with the differential voltage. The encoder 48 generates the measurement signal by converting a comparison result into a digital signal.

The wireless communication circuit 45 converts the measurement signal generated by the analog-to-digital converter 44 into a wireless signal and outputs the converted wireless signal to an external device. The wireless communication circuit 45 includes, for example, a circuit for executing proximity wireless communication such as Blue tooth low energy.

This allows the measurement signal to be output to an external device without restricting human actions. The external device is, for example, a mobile terminal such as a smartphone. The mobile terminal has, for example, a healthcare app installed. The healthcare app calculates the user's heart rate in real time based on the received measurement signal and presents the heart rate to the user. This allows the user to check his or her heart rate in real time while executing some action such as running.

The removal circuit 5 is a circuit that removes a noise signal mixed into the measurement signal. The removal circuit 5 includes an impedance element 51.

The impedance element 51 includes a first end 53 and a second end 54. The first end 53 is connected to each of the ground terminal 52 and the common terminal 43. The second end 54 is connected to the reference terminal 42.

The impedance element 51 is a resistor, a capacitor, an inductor, a diode, or a transistor. The impedance of the impedance element 51 has a value equal to or greater than the human bioimpedance, and preferably has an impedance of 1 to 10 times, preferably 3 to 5 times the bioimpedance. The human bioimpedance is, for example, 1 MΩ.

When the impedance of the impedance element 51 is 0Ω, the ground terminal 52 and the reference terminal 42 are short-circuited.

When the impedance of the impedance element 51 is ∞Ω, the ground terminal 52 and the reference terminal 42 are open.

Since the noise signal is not removed with these configurations as will be described later, in the present embodiment, the impedance of the impedance element 51 is set within a range of greater than 0Ω and less than ∞Ω.

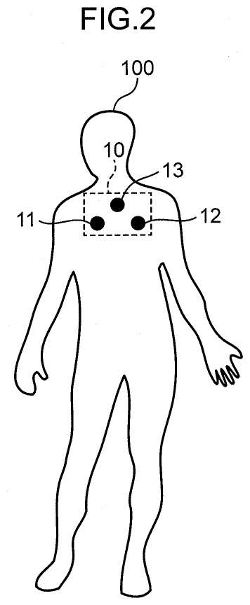

FIG. 2 is a diagram showing an example of attaching the first electrode 11, the second electrode 12, and the ground electrode 13 to a human 100. When measuring the electrocardiographic signal, a measurement area 10 is set, for example, to the body of the human 100. In the example of FIG. 2, the measurement area 10 is set to an area about ¼ of the upper body including the heart. Hereinafter, the left refers to the left direction when the human 100 is viewed from the front, whereas the right refers to the right direction when the human 100 is viewed from the front. The left and right are collectively referred to as a right-and-left direction. The head side of the human 100 is referred to as an upper direction, whereas the foot side of the human 100 is referred to as a lower direction. The upper direction and the lower direction are collectively referred to as an up-and-down direction.

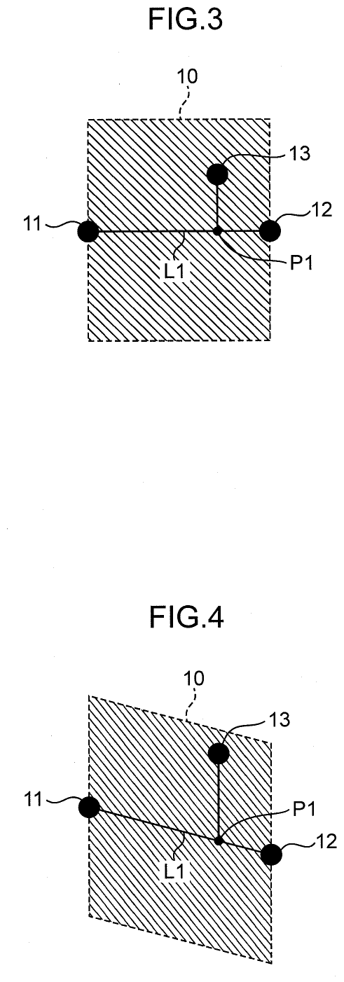

In the measurement area 10, the first electrode 11 is disposed on the left and the second electrode 12 is disposed on the right. The ground electrode 13 is disposed between the first electrode 11 and the second electrode 12 in the measurement area 10. Here, “between the first electrode 11 and the second electrode 12” refers to that, as shown in FIG. 3, a projection point P1 of the ground electrode 13 on a straight line L1 connecting the first electrode 11 and the second electrode 12 is located between the first electrode 11 and the second electrode 12.

FIG. 3 is a diagram showing a first example of the measurement area 10. In the first example, the measurement area 10 is a rectangle. In the example of FIG. 3, the first electrode 11 is disposed at the left end of the measurement area 10, and the second electrode 12 is disposed at the right end of the measurement area 10. The ground electrode 13 is disposed above the straight line L1 connecting the first electrode 11 and the second electrode 12 in the measurement area 10. Note that the ground electrode 13 may be disposed below the straight line L1. The ground electrode 13 may be disposed at the upper end of the measurement area 10 or may be disposed at the lower end of the measurement area 10. The first electrode 11 may be disposed at a place other than the left end as long as within the measurement area 10, and the second electrode 12 may be disposed at a place other than the right end as long as within the measurement area 10.

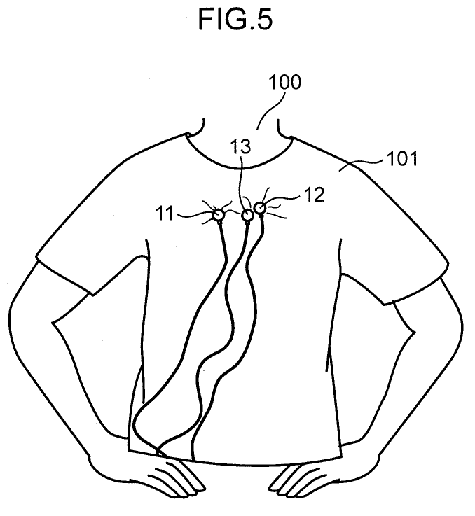

FIG. 4 is a diagram showing a second example of the measurement area 10. In the second example, the measurement area 10 is a parallelogram. In the second example as well, the projection point P1 of the ground electrode 13 with respect to the straight line L1 is located on the segment connecting the first electrode 11 and the second electrode 12, and the ground electrode 13 is disposed between the first electrode 11 and the second electrode 12. The first electrode 11 is disposed above the second electrode 12.

FIG. 5 is a diagram showing an example of mounting the first electrode 11, the second electrode 12, and the ground electrode 13. The first electrode 11, the second electrode 12, and the ground electrode 13 are attached to the chest of an underwear 101. Here, the first electrode 11, the second electrode 12, and the ground electrode 13 are disposed substantially on a straight line. In this example as well, the ground electrode 13 is disposed between the first electrode 11 and the second electrode 12. In this way, by attaching the first electrode 11, the second electrode 12, and the ground electrode 13 to the underwear 101, the human 100 can place the first electrode 11, the second electrode 12, and the ground electrode 13 on the chest only by wearing the underwear 101.

Next, the operation of the measurement device 1 shown in FIG. 1 will be described. The first electrode 11 inputs the first voltage obtained by measuring the human 100 into the input terminal 21 of the differential circuit 2. At the same time, the second electrode 12 inputs the second voltage obtained by measuring the human 100 into the input terminal 22 of the differential circuit 2. The differential circuit 2 generates the differential voltage from the input first voltage and the second voltage, and inputs the differential voltage into the amplifier circuit 3. The amplifier circuit 3 amplifies the input differential voltage and inputs the amplified differential voltage into the analog-to-digital converter 44. The analog-to-digital converter 44 generates the measurement signal by executing analog-to-digital conversion on the input differential voltage, and inputs the measurement signal into the wireless communication circuit 45. The wireless communication circuit 45 converts the measurement signal into a wireless signal and transmits the wireless signal to an external device.

Next, a function of the removal circuit 5 will be described. First, the case C1 in which the impedance of the impedance element 51 is 0Ω will be described. The noise signal that has flowed in from the ground electrode 13 escapes to the ground terminal 52. However, in the case C1, since the ground terminal 52 and the reference terminal 42 are short-circuited, the noise signal is input into the reference terminal 42 without being reduced. This causes the potential of the reference terminal 42 to dynamically fluctuate in conjunction with the noise signal, disabling the analog-to-digital converter 44 from accurately executing analog-to-digital conversion on the differential signal. Note that if the target biosignal is an electrocardiographic signal, then the electromyographic signal, the body surface potential signal, and the internal potential signal are noise signals.

Next, the case C2 in which the impedance of the impedance element 51 is ∞Ω will be described. In the case C2, since the ground terminal 52 and the reference terminal 42 are open, the reference terminal 42 floats from the ground terminal 52. This prevents the ground terminal 52 and the ground electrode 13 from functioning, and prevents the analog-to-digital converter 44 from accurately executing analog-to-digital conversion on the differential signal.

Therefore, in the present embodiment, the impedance of the impedance element 51 is set within a range of greater than 0Ω and less than ∞Ω. Therefore, the noise signal input into the reference terminal 42 described in the case C1 is consumed in the impedance element 51. This allows the potential of the reference terminal 42 to be stable and allows the analog-to-digital converter 44 to accurately execute analog-to-digital conversion on the differential voltage. Furthermore, when the ground electrode 13 is disposed between the first electrode 11 and the second electrode 12 in the measurement device 1, it is confirmed that the accuracy of the biosignal to be measured is more improved than in the case in which the ground electrode is not provided between the first electrode and the second electrode. Therefore, the biosignal to be measured can be measured accurately.

In particular, when the impedance of the impedance element 51 is set at least to a value of the bioimpedance level, the impedance element 51 can consume more noise signal and the noise signal is more accurately removed.

Furthermore, when the impedance of the impedance element 51 is 3 to 10 times, preferably 3 to 5 times the bioimpedance, the impedance element 51 can consume still more noise signal and the noise signal is more accurately removed.

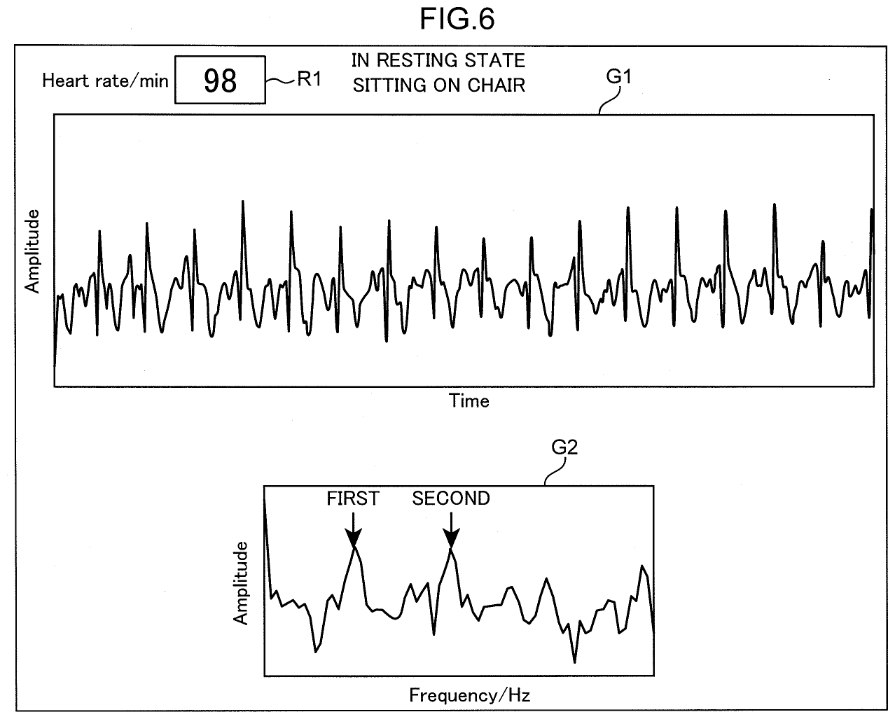

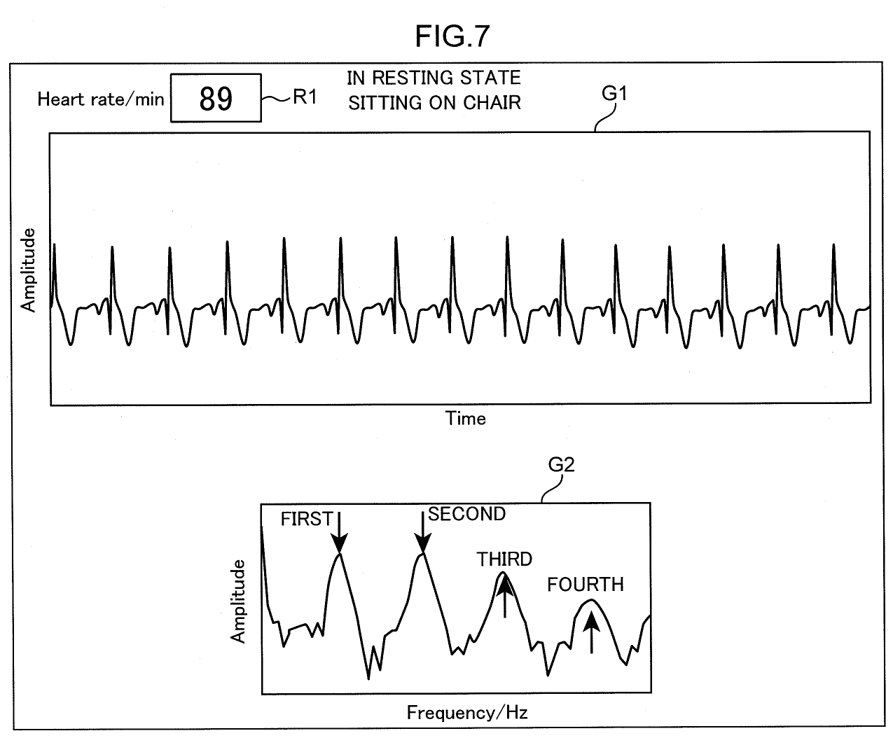

Results of experiments conducted to confirm the effects of the measurement device 1 will be described below. FIG. 6 is a graph showing an experimental result when measuring the electrocardiographic signal of a subject in a resting state by using a measurement device of a comparative example. FIG. 7 is a graph showing an experimental result when measuring the electrocardiographic signal of the subject in a resting state by using the measurement device 1.

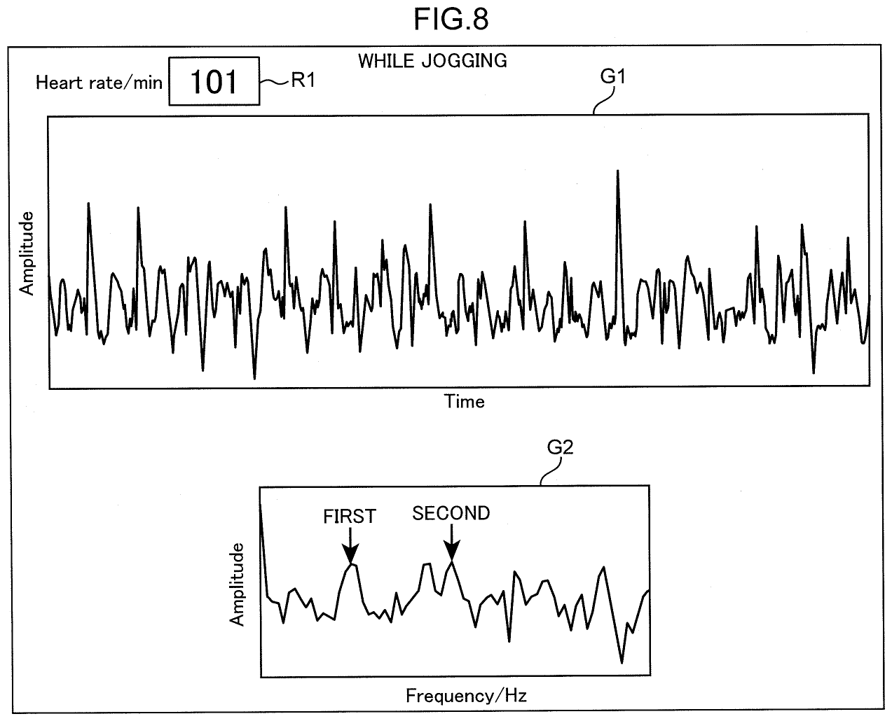

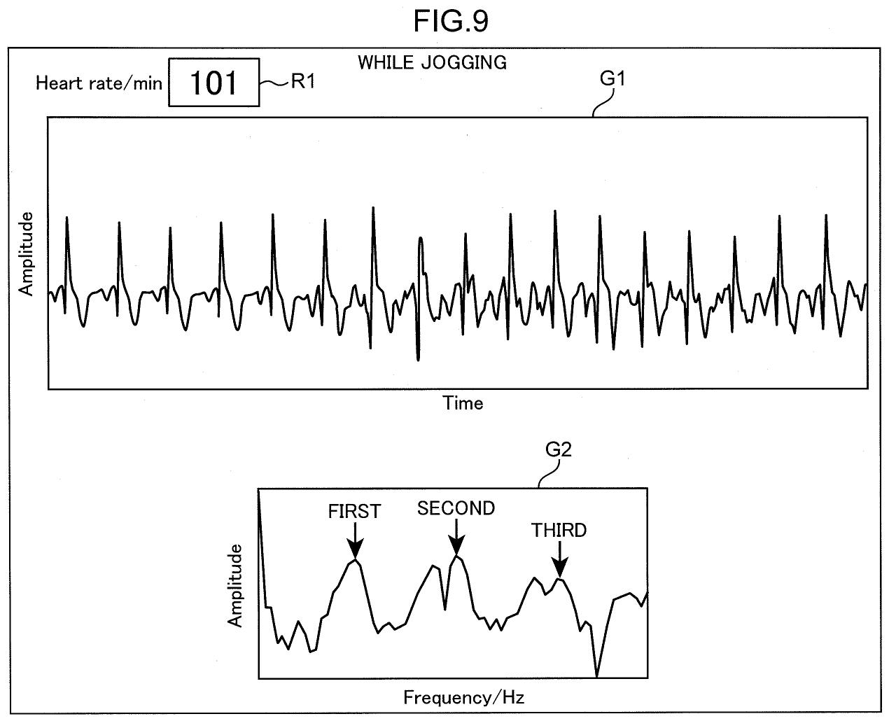

The measurement device of the comparative example has a configuration in which the ground electrode 13 is omitted from the measurement device 1. This is the same for the graphs of FIGS. 8 and 9 as well.

In FIGS. 6 and 7, the graph G1 shows the temporal change of the electrocardiographic signal, with the vertical axis showing amplitude and the horizontal axis showing time. In FIGS. 6 and 7, the graph G2 shows frequency spectra of the electrocardiographic signal, with the vertical axis showing amplitude and the horizontal axis showing frequency. In FIGS. 6 and 7, the column R1 shows the heart rate per minute obtained from the electrocardiographic signal. This is the same for the graphs of FIGS. 8 and 9 as well.

The measurement device of the comparative example measures only the electrocardiographic signal up to the first and second harmonics as shown in the graph G2 of FIG. 6. In contrast, the measurement device 1 measures the electrocardiographic signal from the first harmonic to the fourth harmonic as shown in the graph G2 of FIG. 7. Therefore, it is confirmed that the measurement device 1 can measure the electrocardiographic signal more accurately than the measurement device of the comparative example.

FIG. 8 is a graph showing an experimental result when measuring the electrocardiographic signal of the subject in a jogging state by using the measurement device of the comparative example. FIG. 9 is a graph showing an experimental result when measuring the electrocardiographic signal of the subject in a jogging state by using the measurement device 1.

The measurement device of the comparative example measures only the electrocardiographic signal up to the first and second harmonics as shown in the graph G2 of FIG. 8. In contrast, the measurement device 1 measures the electrocardiographic signal from the first harmonic to the third harmonic as shown in the graph G2 of FIG. 9. Therefore, it is confirmed that the measurement device 1 can measure the electrocardiographic signal more accurately than the measurement device of the comparative example. When a resting state and a jogging state are compared, it is confirmed that the electrocardiographic signal can be measured more accurately in a resting state.

Next, an experimental result conducted to confirm the influence of the impedance value of the impedance element 51 on the measurement accuracy will be described.

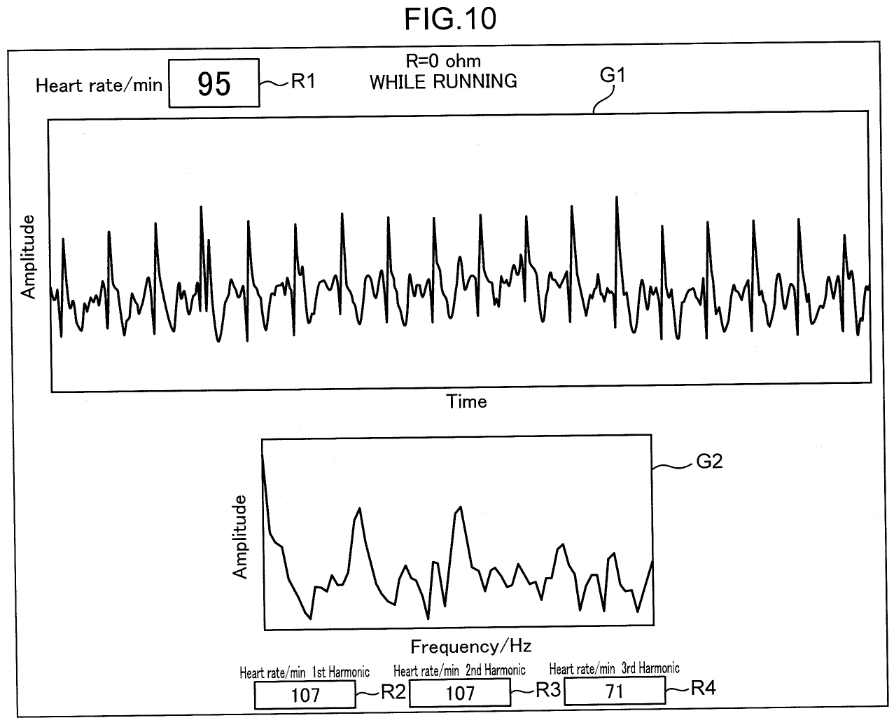

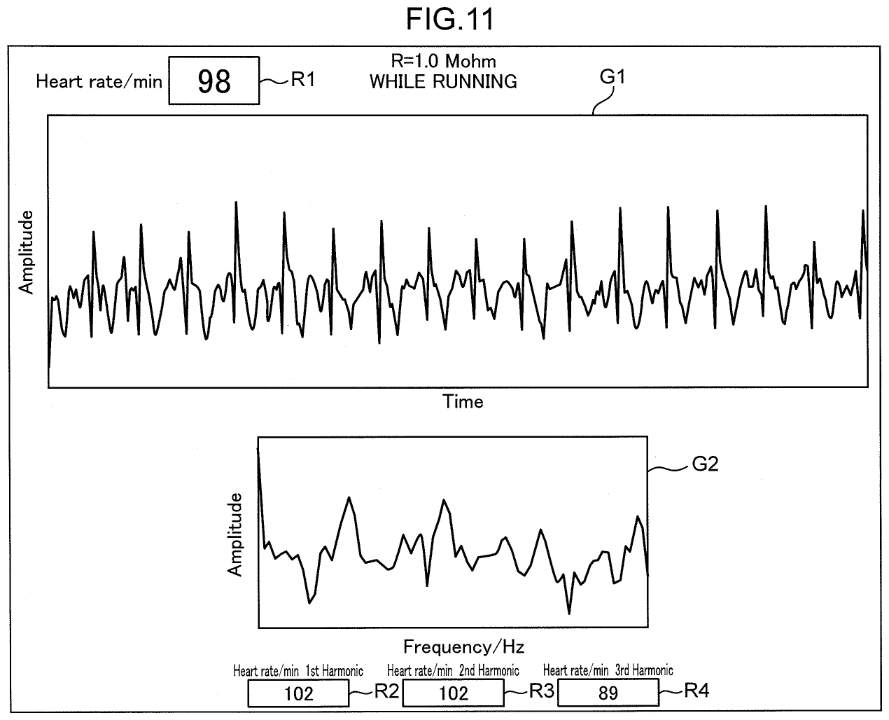

FIG. 10 is a graph showing an experimental result when measuring the electrocardiographic signal of the subject in a running state by using the measurement device 1 with the impedance of the impedance element 51 of 0Ω. In FIG. 10, the graph G1 shows the temporal change of the electrocardiographic signal, the vertical axis shows amplitude, the horizontal axis shows time, the graph G2 shows the frequency spectrum of the electrocardiographic signal, the vertical axis shows amplitude, and the horizontal axis shows frequency. The column R1 shows the heart rate per minute obtained from the electrocardiographic signal, the column R2 shows the heart rate per minute obtained from the first harmonic of the electrocardiographic signal, the column R3 shows the heart rate per minute obtained from the second harmonic of the electrocardiographic signal, and the column R4 shows the heart rate per minute obtained from the third harmonic of the electrocardiographic signal. This is the same for the graphs of FIGS. 11 to 17 as well.

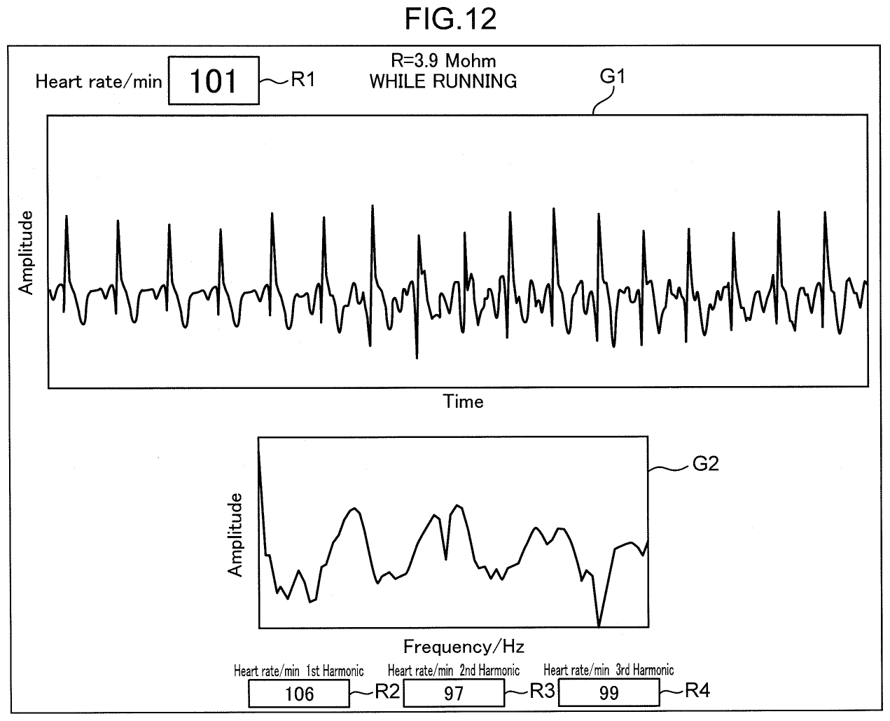

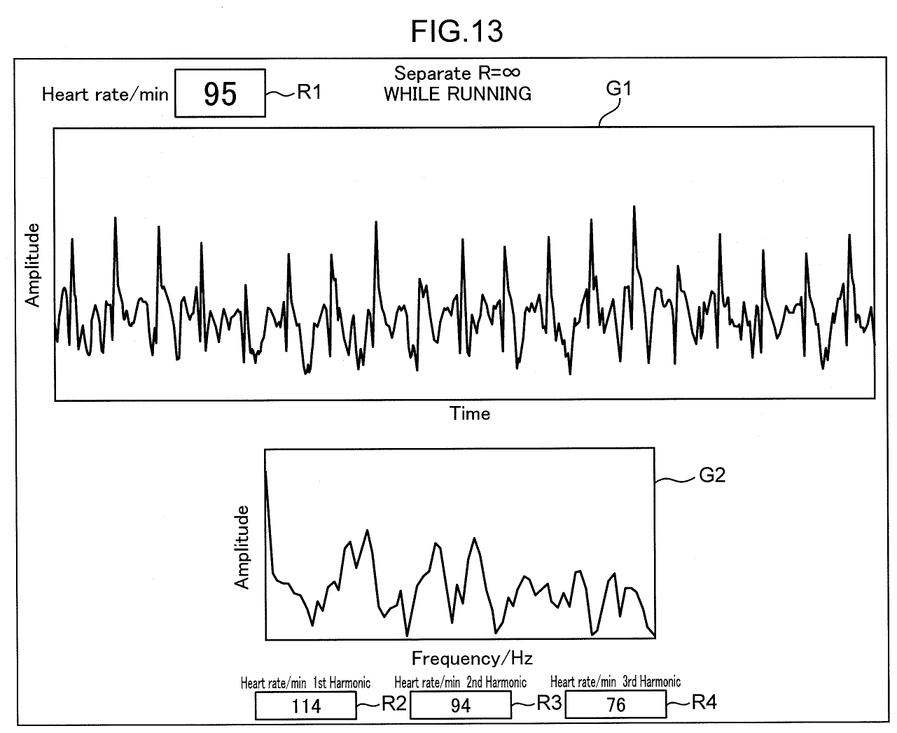

FIG. 11 is a graph showing an experimental result when measuring the electrocardiographic signal of the subject in a running state by using the measurement device 1 with the impedance of the impedance element 51 of 1.0 MΩ. FIG. 12 is a graph showing an experimental result when measuring the electrocardiographic signal of the subject in a running state by using the measurement device 1 with the impedance of the impedance element 51 of 3.9 MΩ. FIG. 13 is a graph showing an experimental result when measuring the electrocardiographic signal of the subject in a running state by using the measurement device 1 with the impedance of the impedance element 51 of ∞Ω.

Comparing FIGS. 10 to 13, the heart rate of the third harmonic per minute is “71” when the impedance is 0 MΩ, is “89” when the impedance is 1 MΩ, is “99” when the impedance is 3.9 MΩ, and is “76” when the impedance is ∞Ω. Therefore, the amplitude of the third harmonic is maximum when the impedance is 3.9 MΩ, is next when the impedance is 1 MΩ, is next when the impedance is ∞Ω, and is next when the impedance is 0 MΩ. It is confirmed that if the impedance is about 3.9 times the bioimpedance, the measurement accuracy of the electrocardiographic signal increases because the bioimpedance of the subject is approximately 1 MΩ. It is confirmed that the heart rate of the third harmonic per minute decreases when the impedance is significantly smaller than the bioimpedance or the impedance is significantly larger than the bioimpedance, and that the measurement accuracy of the electrocardiographic signal decreases when the impedance is ∞Ω.

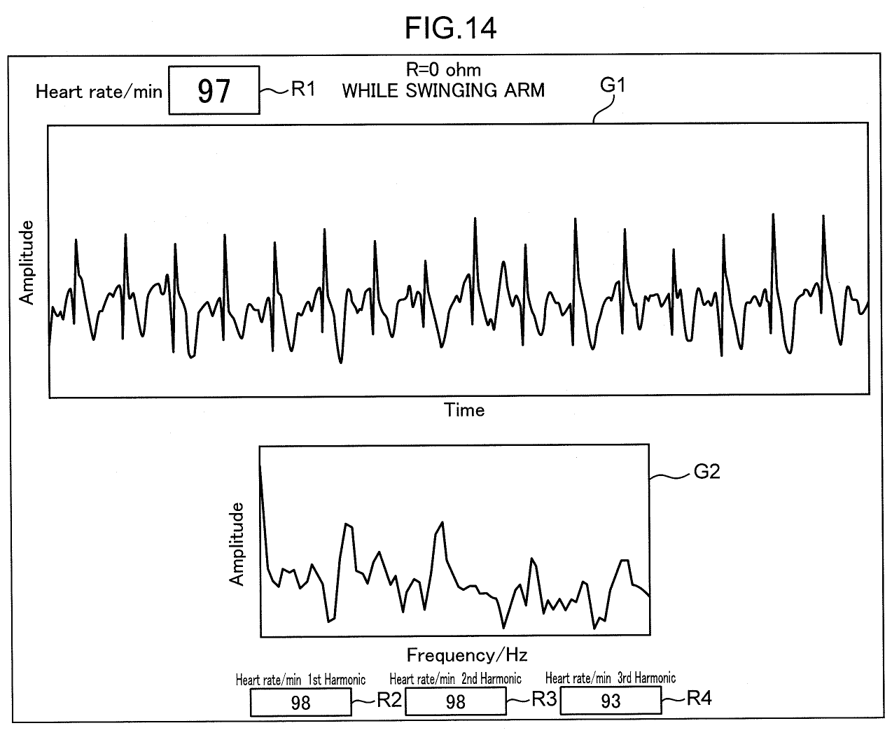

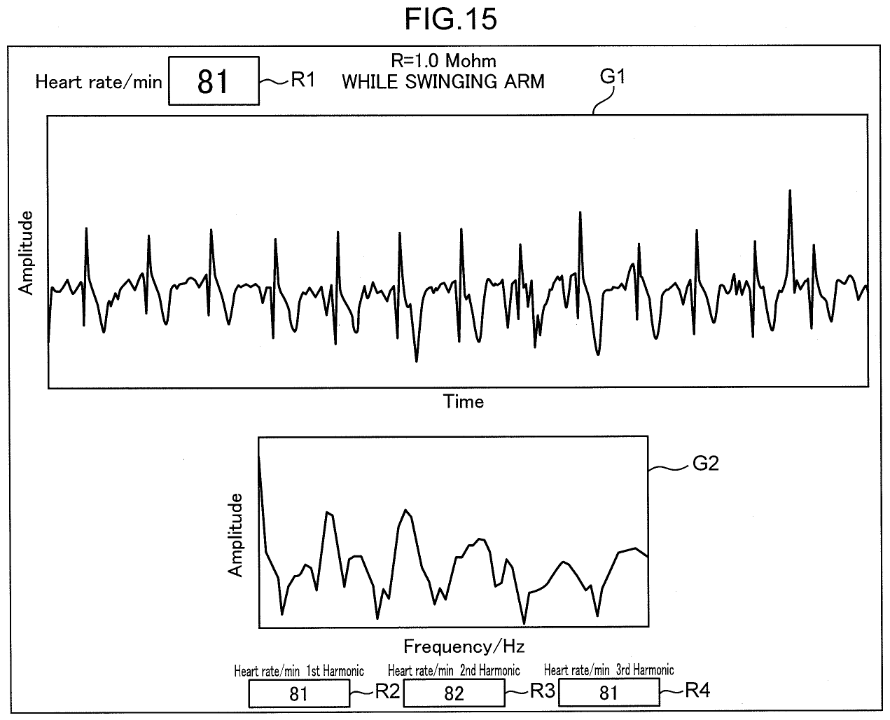

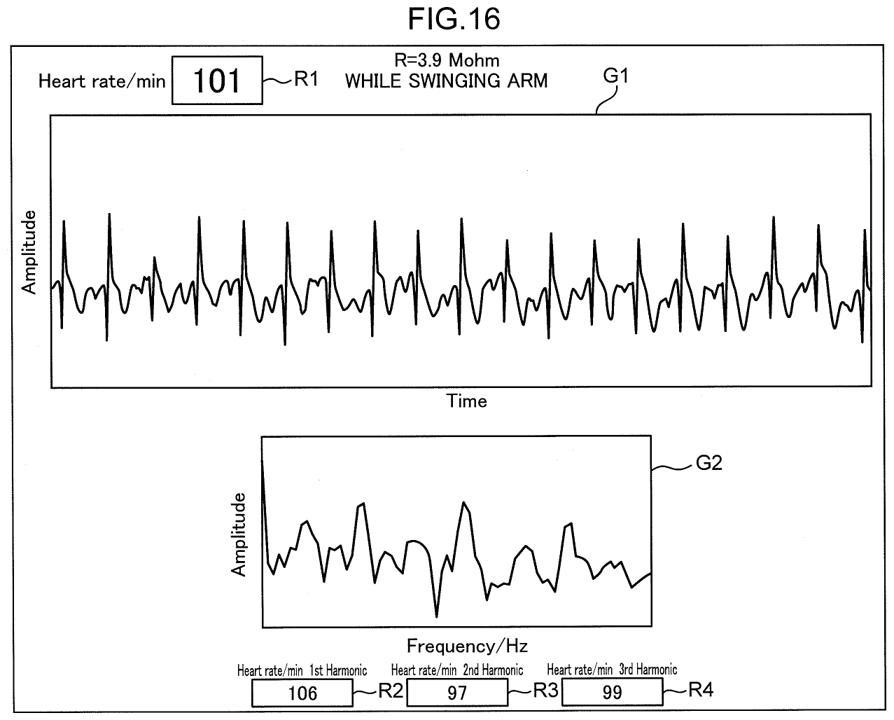

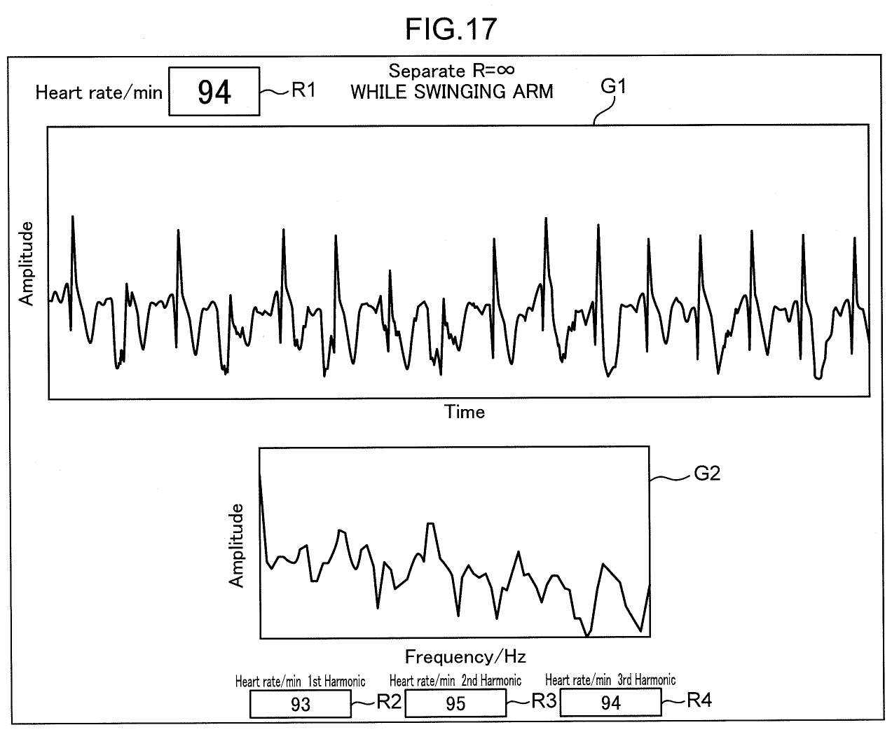

FIG. 14 is a graph showing an experimental result when measuring the electrocardiographic signal of the subject in an arm-swinging state by using the measurement device 1 with the impedance of the impedance element 51 of 0Ω. FIG. 15 is a graph showing an experimental result when measuring the electrocardiographic signal of the subject in an arm-swinging state by using the measurement device 1 with the impedance of the impedance element 51 of 1.0Ω. FIG. 16 is a graph showing an experimental result when measuring the electrocardiographic signal of the subject in an arm-swinging state by using the measurement device 1 with the impedance of the impedance element 51 of 3.9 MΩ. FIG. 17 is a graph showing an experimental result when measuring the electrocardiographic signal of the subject in an arm-swinging state by using the measurement device 1 with the impedance of the impedance element 51 of ∞Ω.

Comparing FIGS. 14 to 17, the heart rate of the third harmonic per minute is “93” when the impedance is 0Ω, is “81” when the impedance is 1 MΩ, is “97” when the impedance is 3.9 MΩ, and is “94” when the impedance is ∞Ω. Therefore, the heart rate of the third harmonic per minute is maximum when the impedance is 3.9 MΩ, is next when the impedance is ∞MΩ, is next when the impedance is 0 MΩ, and is next when the impedance is 1 MΩ. The heart rate of the first harmonic per minute is larger when the impedance is 3.9 MΩ than when ∞Ω. Therefore, it is confirmed that the electrocardiographic signal has good accuracy when the impedance is 3.9 MΩ.

The present invention can employ the following modifications.
(1) When the measurement signal is an electroencephalographic signal, the measurement area 10 can be set to the head of the human 100.
(2) When the measurement signal is an electromyographic signal, the measurement area 10 can be set to an area including the muscle part to be measured. For example, when measuring the electromyographic signal of the calf or thigh, the measurement area 10 is set to the calf or thigh. When measuring the electromyographic signal of the arm, the measurement area 10 is set to the arm.
(3) The measurement area 10 may be the chest or the back of the body of the human 100. FIG. 18 is a diagram showing an example of attaching the first electrode 11, the second electrode 12, and the ground electrode 13 when the measurement area 10 is the back. In FIG. 18, when the human 100 is viewed from the back, the right direction is referred to as the right, and the left direction is referred to as the left. In the example of FIG. 18, the measurement area 10 is set to the center of the back, but this is one example. The measurement area 10 may be a place other than the center, such as the upper or lower portion of the back. In the measurement area 10, the first electrode 11 is disposed on the left and the second electrode 12 is disposed on the right. The ground electrode 13 is disposed between the first electrode 11 and the second electrode 12 in the measurement area 10. “Between the first electrode 11 and the second electrode 12” is as described in FIG. 3.

In this way, when the first electrode 11, the second electrode 12, and the ground electrode 13 are disposed on the back, it is possible to measure the electrocardiographic signal that allows the heart rate and the respiratory rate to be easily counted. It is difficult for the human 100 such as a person with dementia or a person requiring care to remove the first electrode 11, the second electrode 12, and the ground electrode 13 from the back, enabling continuous heart rate measurement. Furthermore, a caregiver can easily attach the first electrode 11, the second electrode 12, and the ground electrode 13 to the human 100 who requires nursing care.
(4) The first electrode 11, the second electrode 12, and the ground electrode 13 may not be directly attached to the human 100.

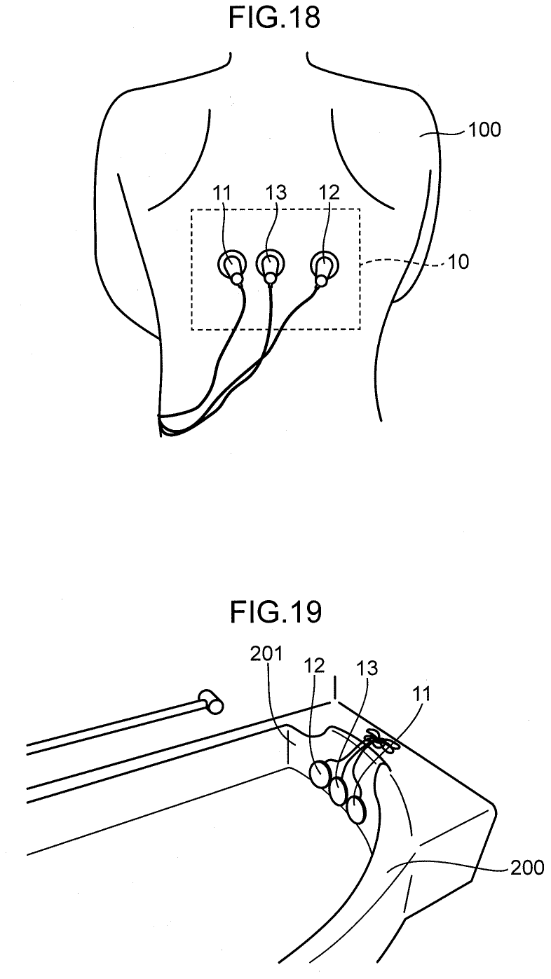

FIG. 19 is a diagram showing the first electrode 11, the second electrode 12, and the ground electrode 13 attached to a bathtub 200. The bathtub 200 includes an inner surface 201. The inner surface 201 is an inner surface located on the head side of the human 100 when the human 100 is lying down in the bathtub 200. The first electrode 11, the second electrode 12, and the ground electrode 13 are attached to the inner surface 201 side by side in the right-and-left direction (lateral direction). In more detail, the ground electrode 13 is attached between the first electrode 11 and the second electrode 12.

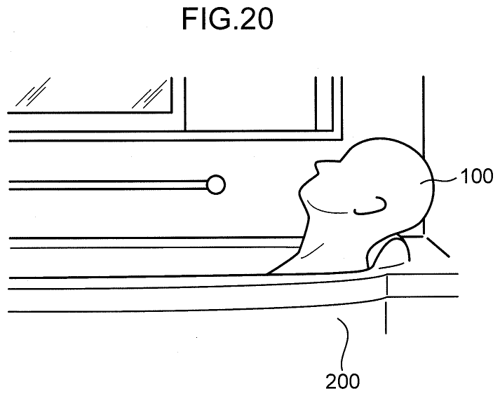

FIG. 20 is a diagram showing the human 100 taking a bath in the bathtub 200. When the human 100 lies down in the bathtub 200 with the back abutting the inner surface 201, the back of the human 100 will abut the first electrode 11, the second electrode 12, and the ground electrode 13. As a result, the measurement device 1 can measure the electrocardiographic waveform of the human 100 in a bathed and relaxed state.

Note that in the present embodiment, “the first electrode 11, the second electrode 12, and the ground electrode 13 are disposed in the body measurement area” includes not only the aspect in which the first electrode 11, the second electrode 12, and the ground electrode 13 are directly attached to the human 100, but also the aspect in which the first electrode 11, the second electrode 12, and the ground electrode 13 abut the body as a result of the human 100 abutting the body to an object to which the first electrode 11, the second electrode 12, and the ground electrode 13 are attached.

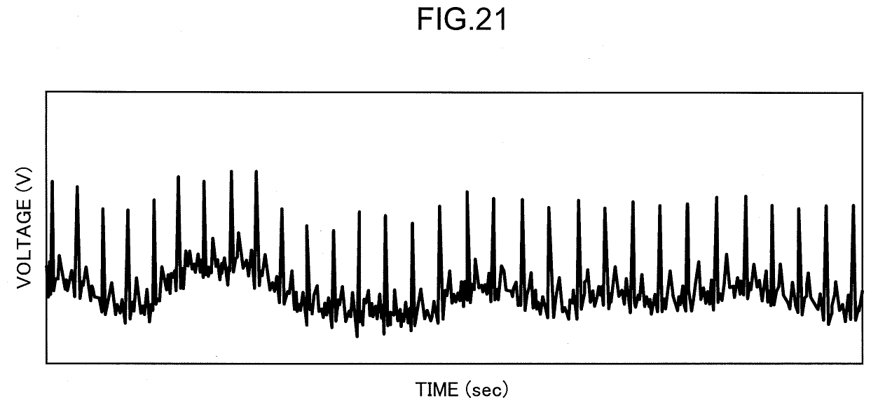

FIG. 21 is a diagram showing the electrocardiographic signal of the human 100 while taking a bath. In FIG. 21, the vertical axis shows voltage and the horizontal axis shows time. As shown in FIG. 21, by disposing the first electrode 11, the second electrode 12, and the ground electrode 13 disposed in the bathtub 200, it is confirmed that the electrocardiographic signal in which the P wave, the R wave, and the T wave appear well can be measured.

### Summary of Embodiments

Technical features of the embodiments are summarized below.

A measurement device according to one aspect of the present invention is a measurement device for measuring a biosignal, including: a first electrode disposed in a measurement area of a body according to the biosignal to be measured; a second electrode disposed in the measurement area; a ground electrode disposed between the first electrode and the second electrode in the measurement area; a differential circuit configured to generate a differential voltage between a first voltage input from the first electrode and a second voltage input from the second electrode; an output circuit including an input terminal to which the differential voltage is input, a reference terminal to which a reference voltage is input, and a common terminal, the output circuit being configured to generate a measurement signal based on the differential voltage and the reference voltage, and to output the generated measurement signal; a ground terminal connected to each of the ground electrode, the differential circuit, and the output circuit; and an impedance element including a first end connected to each of the ground terminal and the common terminal, and a second end connected to the reference terminal.

With this configuration, the ground electrode disposed between the first electrode and the second electrode plays the role of letting a noise signal including the biosignal that is not to be measured escape to the ground terminal. Furthermore, with this configuration, the impedance element is provided with the first end connected to each of the ground terminal and the common terminal, and the second end connected to the reference terminal. Therefore, even if the noise signal escaped to the ground terminal flows backward toward the reference terminal, this backflow noise signal is consumed by the impedance element. This prevents the noise signal from being mixed into the reference terminal, and allows the noise signal to be removed accurately. When the ground electrode is disposed between the first electrode and the second electrode in the measurement area of the body according to the biosignal to be measured, it is confirmed that the accuracy of the biosignal to be measured is improved more than in the case where the ground electrode is not provided between the first electrode and the second electrode. Therefore, the biosignal to be measured can be measured accurately.

Furthermore, since such a configuration removes the noise signal, there is no need to separately provide a signal processing circuit, and it is possible to reduce the size and cost of the device.

In the above measurement device, the impedance element may have an impedance equal to or greater than a bioimpedance of a living body.

With this configuration, since the impedance element has the impedance equal to or greater than the bioimpedance, the noise signal can be removed immediately after the measurement starts.

In the above measurement device, the impedance of the impedance element may be 3 to 10 times the bioimpedance.

With this configuration, since the impedance element has the impedance 3 to 10 times the bioimpedance, the noise signal can be removed immediately after the measurement starts.

In the above measurement device, the biosignal to be measured may be an electrocardiographic signal, an electroencephalographic signal, or an electromyographic signal.

With this configuration, the electrocardiographic signal, the electroencephalographic signal, or the electromyographic signal can be accurately measured.

In the above measurement device, the impedance element may be a resistor, a capacitor, an inductor, a diode, or a transistor.

With this configuration, since the impedance element is a resistor, a capacitor, an inductor, a diode, or a transistor, the impedance element can be configured simply and at low costs by using an existing circuit element.

In the above measurement device, the output circuit may include an analog-to-digital converter configured to convert the differential voltage into a digital signal based on the differential voltage and the reference voltage, and to output the converted digital signal as the measurement signal.

With this configuration, since the measurement signal is generated by converting the differential voltage into a digital signal, the measurement signal can be output to an external device accurately.

In the above measurement device, the output circuit may include a wireless communication circuit configured to convert the measurement signal into a wireless signal and to output the wireless signal to an external device.

With this configuration, since the measurement signal is converted into a wireless signal and output to an external device, the measurement signal can be output to an external device without restricting the action of the living body.

The above measurement device may further include an amplifier circuit configured to amplify the differential voltage and input the amplified differential voltage into the output circuit.

With this configuration, since the differential voltage is amplified from the differential circuit, the amplitude of the differential voltage can be adjusted to amplitude suitable for the output circuit to generate the measurement signal.

In the above measurement device, the measurement area may be a back.

With this configuration, it is possible to obtain the electrocardiographic signal that allows the heart rate and the respiratory rate to be easily counted.

In the above measurement device, the first electrode, the second electrode, and the ground electrode may be attached to an inner surface of a bathtub.

With this configuration, it is possible to measure the measurement signal of a human in a relaxed state while taking a bath.

1. A measurement device for measuring a biosignal, the measurement device comprising:
a first electrode disposed in a measurement area of a body according to the biosignal to be measured;
a second electrode disposed in the measurement area;
a ground electrode disposed between the first electrode and the second electrode in the measurement area;
a differential circuit configured to generate a differential voltage between a first voltage input from the first electrode and a second voltage input from the second electrode;
an output circuit including an input terminal to which the differential voltage is input, a reference terminal to which a reference voltage is input, and a common terminal, the output circuit being configured to generate a measurement signal based on the differential voltage and the reference voltage, and to output the generated measurement signal;
a ground terminal connected to each of the ground electrode, the differential circuit, and the output circuit; and
an impedance element including a first end connected to each of the ground terminal and the common terminal, and a second end connected to the reference terminal.

2. The measurement device according to claim 1, wherein
the impedance element has an impedance equal to or greater than a bioimpedance of a living body.

3. The measurement device according to claim 2, wherein
the impedance of the impedance element is 3 to 10 times the bioimpedance.

4. The measurement device according to claim 1, wherein
the biosignal to be measured is an electrocardiographic signal, an electroencephalographic signal, or an electromyographic signal.

5. The measurement device according to claim 1, wherein
the impedance element is a resistor, a capacitor, an inductor, a diode, or a transistor.

6. The measurement device according to claim 1, wherein
the output circuit includes an analog-to-digital converter configured to convert the differential voltage into a digital signal based on the differential voltage and the reference voltage, and to output the converted digital signal as the measurement signal.

7. The measurement device according to claim 1, wherein
the output circuit includes a wireless communication circuit configured to convert the measurement signal into a wireless signal and to output the wireless signal to an external device.

8. The measurement device according to claim 1, further comprising an amplifier circuit configured to amplify the differential voltage and input the amplified differential voltage into the output circuit.

9. The measurement device according to claim 1, wherein
the measurement area is a back.

10. The measurement device according to claim 1, wherein
the first electrode, the second electrode, and the ground electrode are attached to an inner surface of a bathtub.
